(19) United States
(12) Patent Application Publication
(10) Pub. No.: US 2026/0108161 A1
(43) Pub. Date: Apr. 23, 2026

(54) FLEXIBLE DUAL-MODALITY SYSTEM AND METHOD FOR CONTINUOUS BLOOD PRESSURE MONITORING

(71) Applicants: The Chinese University of Hong Kong, Hong Kong (CN); Hong Kong Centre for Cerebro-Cardiovascular Health Engineering Limited, Hong Kong (CN)

(72) Inventors: Ni ZHAO, Hong Kong (CN); Cunman LIANG, Tangshan (CN); Zhou JIANG, Hong Kong (CN); Shirong QIU, Meizhou (CN); Yuan-Ting ZHANG, Hong Kong (CN)

(21) Appl. No.: 19/366,675

(22) Filed: Oct. 23, 2025

Related U.S. Application Data
(60) Provisional application No. 63/710,715, filed on Oct. 23, 2024.

(51) Int. Cl. A61B 5/021; A61B 5/00; A61B 5/02

(52) U.S. Cl. CPC A61B 5/02141; A61B 5/02007; A61B 5/02125; A61B 5/7275; A61B 2560/0468; A61B 2562/0204; A61B 2562/0247; A61B 2562/066; A61B 2562/164; A61B 2562/18

(57) ABSTRACT

A flexible dual-modality system and methods for continuous blood pressure monitoring are provided. The system includes a first layer including a skin friendly contact layer, a pulse waveform sensor, and an ultrasound transducer common ground electrode layer, each set of pulse waveform sensors including two pressure sensors; a second layer including a cavity, a copper cube, an ultrasound transducer array, a capsulation material; and a third layer including an ultrasound transducer stimulating electrode layer, and a skin friendly contact layer. The method includes extracting blood vessel parameters from signals measured by a dual-modality device; combining the basic vascular parameters to determine individual vascular parameters; and determining comprehensive BP-related vascular characteristic parameters from the individual vascular parameters, for providing a vascular profile related to blood pressure. The flexible and layered architecture can be configured to improve manufacturability, effectiveness, compactness, and usability of the blood pressure monitoring method and systems in clinical applications.

### CROSS-REFERENCE TO RELATED APPLICATION

The present application claims the benefit of U.S. Provisional Application Ser. No. 63/710,715, filed Oct. 23, 2024, which is hereby incorporated by reference herein in its entirety, including any figures, tables, or drawings.

### BACKGROUND OF THE INVENTION

Cardiovascular diseases are globally recognized as the leading cause of death, taking approximately 18 million lives each year. These diseases are characterized by high morbidity and mortality rates, and the timeliness of treatment is much higher than that of other diseases. Therefore, the prevention and early detection of cardiovascular diseases are of paramount importance.

Blood pressure measurement plays a vital role in the prevention, early detection, and management of cardiovascular diseases.

Accurate blood pressure measurement provides essential information for healthcare professionals to assess cardiovascular health and make informed decisions regarding treatment strategies. This information is vital for determining the appropriate medication dosage, evaluating the effectiveness of prescribed treatments, and adjusting treatment plans as needed. Clinical and commercial applications of blood pressure measurement are extensive, encompassing both medical settings and home health monitoring. In medical settings, blood pressure measurement systems are widely used for diagnosing and monitoring a range of diseases [1].

Traditional passive, unobstructed and continuous cuffless blood pressure (BP) measurement methods have attracted significant attention in both research and industry. These methods encompass a variety of techniques, including pressure sensor-based tonometry, photoplethysmography (PPG), bioimpedance and bioelectrical measurements, as well as acoustic methods such as ultrasound wall-tracking. Despite their potential, these traditional wearable BP monitoring methods face several key limitations that have hindered their broader adoption:
1) Frequent calibration issues, particularly the need for individual calibration to account for physiological differences between users. This makes the initial setup and ongoing maintenance of these devices burdensome for both clinicians and consumers.
2) Poor accuracy and instability over time, because the accuracy of BP estimation algorithm declines with the performance of device when being used for prolonged periods of time, especially for poor device-skin interface, functional inadequacy and insufficient power supply. This undermines confidence in the BP readings, making it difficult to detect meaningful changes in a person's blood pressure.
3) Difficulty in adapting to distinct measurement scenarios and diverse populations, such as variations in skin tone, body habitus, motion artifacts and vascular characteristics.

Specifically, the single-mode sensors often encounter above-described problems, leading to inconsistent performance across users and conditions.

Nevertheless, comprehensive analysis of blood pressure-related vascular characteristics is essential for accurate BP measurement. This process requires extraction of detailed information about an individual's vascular system, including various hemodynamic and structural parameters.

From a hemodynamic perspective, measuring changes in arterial stiffness is crucial, as it serves as a key indicator of cardiovascular health and a major determinant of blood pressure. Metrics such as pulse wave velocity and augmentation index provide insights into the dynamic properties of the arteries and how they respond to the pulsatile flow of blood. These hemodynamic biomarkers can reveal underlying conditions such as atherosclerosis or hypertension that affect blood pressure regulation.

Additionally, precise characterization of the blood vessel geometry is essential, including measurements of arterial diameters, cross-sectional areas, and distensibility of arteries at multiple points throughout the vascular tree. Since the size and compliance of these vessels directly influence pressure wave propagation and the overall hemodynamics that govern blood pressure, variations in these structural vascular parameters between individuals are one of major sources of the inconsistencies observed with traditional single-mode cuffless BP devices.

Regarding related art designs stretchable ultrasound transducer devices, Xu et al., (U.S. patent application Ser. No. 16/477,060) describes a conformable piezoelectric transducer array for performing ultrasound includes a silicone elastomer substrate and a silicone elastomer superstrate [2]. However, due to this device relies solely on ultrasound to measure diameter change of the artery, it requires constant calibration when measuring different individuals or at different times for accurate blood pressure measurements.

Related art includes a system for calibration-free, cuffless blood pressure evaluation. Mashood et al. (U.S. Patent Application No. 2021/0077057) focuses on blood pressure measurement using an arterial compliance probe [3]. This system includes an ultrasound-based probes and pressure or force sensors or bio-potential transducers. The ultrasound transducers are configured to measure a change in arterial dimensions, pulse wave velocity, and other character traits of an arterial segment over continuous cardiac cycle, which is then used to evaluate blood pressure parameters. The pressure sensor/force sensor/bio-potential transducers are configured to measure the mechanical and electrical signals in arteries, which are used to evaluate arterial blood pressure and stiffness indices. However, the patent content solely encompasses a conceptual design and does not delve into the precise layout of the dual-mode blood pressure measurement device. In the referenced patent, the change in arterial dimensions and the pulse wave velocity are both measured with the same ultrasound sensors, which results in the measured signals not being in the same pulse cycle. As the arterial parameters measured during different pulse cycles often change, the aforementioned use of ultrasound sensor for two parameter recording will reduce the accuracy of blood pressure assessment. The referenced patent proposes a handheld arterial compliance probe. This device is usually heavy, rigid and bulky, which is not suitable for long-term and real-time monitoring of blood pressure, and do not have a solid interfacial contact and therefore good coupling with irregular nonplanar surfaces.

Therefore, there is a pressing need to develop a dual-modality approach that comprehensively monitors both hemodynamic and structural parameters of an individual's unique vascular profile, offering potentials for cuffless blood pressure monitoring that rival clinical-grade cuff-based methods.

### BRIEF SUMMARY OF THE INVENTION

There continues to be a need in the art for improved designs and techniques for flexible dual-modality system and method for continuous blood pressure monitoring.

According to an embodiment of the subject invention, a dual-modality method for measuring cardiovascular parameters and extracting blood pressure (BP)-related vascular characteristics is provided. The method comprises extracting blood vessel parameters from signals measured by a dual-modality device; combining the basic vascular parameters to determine individual vascular parameters; and determining comprehensive BP-related vascular characteristic parameters from the individual vascular parameters, for providing a vascular profile related to blood pressure. The blood vessel parameters include a dynamic vessel diameter, a cross-sectional area of vessels, and arterial stiffness. The individual vascular parameters, include pulse wave velocities, pulse stiffening ratios, and dynamic beta stiffness indexes. The BP-related vascular characteristic parameters are configured to enable individual calibration-free BP measurement and continuous BP tracking. Moreover, the dual-modality device includes both ultrasound and pulse sensors to enable stable and reliable extraction of vascular data to support a cuffless BP method. The method may further comprise cuffless and calibration-free blood pressure (BP) steps including BP-related vascular characterization extraction steps; and pulse time decay compensation steps. Each of the ultrasound sensors includes one element or a plurality of elements and the pulse sensors include a pressure-based sensor, and/or a plethysmography-based sensor. The BP-related vascular characterization extraction steps extract features from the pulse signals and ultrasound signals by the dual-modality device. Furthermore, the BP-related vascular characterization extraction steps enable the characteristic cuffless BP measurement based on the pulse wave velocities, blood vessel diameters, and cross-sectional blood vessel areas for individual static cuffless BP measurement. The BP-related vascular characterization extraction steps enable different types of arterial stiffness parameters calculation based on the pulse wave velocities, the pulse stiffening ratios, and the beta stiffness indexes. In addition, the BP-related vascular characterization extraction steps enable accurate BP monitoring by combing the static cuffless BP measurement with the dynamic beta stiffness. The pulse time decay compensation steps are based on consistent trend between pulse waveform time decay and BP time decay and is configured by combining real-time pulse time decay and individual dynamic blood vessel diameters to enable continuous and accurate BP trend tracking. The pulse time decay BP compensation steps are configured to extract features from the pulse signals and ultrasound signals. The pulse time decay BP compensation steps are configured to calculate the time decay of BP based on the pulse waveforms. The pulse time decay BP compensation steps are configured to calculate the compensated BP based on the time decay of BP. The pulse time decay BP compensation steps are configured to enable accurately continuous BP measurement by combining the compensated BP and the characteristic cuffless BP.

In certain embodiments of the subject invention, a flexible dual-modality system for continuous blood pressure monitoring is provided. The system comprises a first layer (401) comprising a skin friendly contact layer (410), a pulse waveform sensor (420), and an ultrasound transducer common ground electrode layer (430), wherein each set of pulse waveform sensors comprise two pressure sensors; a second layer (402) comprising a cavity (440), a copper cube (450), an ultrasound transducer array (460), a capsulation material (Ecoflex) (470); and a third layer (403) comprising an ultrasound transducer stimulating electrode layer (480), and a skin friendly contact layer (490). The dual-modality system may further comprise at least two sets of pulse waveform sensors, two cavities, eight pixels, two skin friendly contact layers, and two electrode layers. The skin friendly contact (410) layer comprises a silicone rubber, Ecoflex, PDMS (polydimethylsiloxane), or SEBS, to provide biocompatibility, flexibility, and durability. The dual-modality system may further comprise two electrodes. The two electrodes are made from at least one material selected from gold (Au), silver (Ag), copper (Cu), another conductive metal, a conductive polymer, and a conductive non-metal, non-polymer material. The two electrodes are formed to have a serpentine structure for stretchability. Moreover, the ultrasound sensing unit comprises ultrasound transducers (460) and copper cube (450). The ultrasound transducer is formed of piezoelectric ceramics with 1-3 composite structure for performance in d₃₃ direction to improved acoustic coupling and electromechanical conversion, allowing enhanced ultrasound signal detection and transmission capabilities. The copper cube (150) has same thickness as that of the ultrasound transducer, allowing smooth routing of common ground electrode towards a same plane as that of the stimulating electrode. The pulse waveform sensing unit comprises a pressure sensor and a cavity. In addition, the pressure sensor is made by organic flexible piezoelectric materials including polyvinylidene difluoride (PVDF) and has a serpentine structure to withstand bending and tensile deformation. The cavity is made by a high stiffness silicon rubber including PDMS, the cavity is located on the rectangular pressure sensor beam to form a piezo cantilever to improve force sensitivity of the pressure sensor. Furthermore, the entire system is encapsulated by a silicone rubber with modulus on par with that of human skin to provide a barrier to moisture for protecting the system from sweat corrosion, to allow conformation to different surfaces, and to endure twisting and stretching.

### BRIEF DESCRIPTION OF THE DRAWINGS

FIG. 1A is a schematic representation of an example element arrangement of the flexible and stretchable dual-modality device with one central ultrasound sensor and two-end pulse sensors; FIG. 1B is a schematic representation of an example element arrangement of the flexible and stretchable dual-modality device with multi-element of central ultrasound sensors and two-end pulse sensors; FIG. 1C is a schematic representation of an example element arrangement of the flexible and stretchable dual-modality device with multi-element of central ultrasound sensors and multi-element of two-end pulse sensors, according to an embodiment of the subject invention.

FIG. 2 illustrates the physiological structure and dual-modality device interaction with the tissue, according to an embodiment of the subject invention.

FIG. 3 illustrates the framework of the cuffless blood pressure (BP) measurement (100), including the hardware and software design, according to an embodiment of the subject invention.

FIG. 4 illustrates the BP-related vascular characterization extraction algorithm for cuffless BP measurement, according to an embodiment of the subject invention.

FIG. 5 illustrates the pulse time decay BP compensation strategy, according to an embodiment of the subject invention.

FIG. 6 illustrate a layout of a dual-modality device (400) comparing two types of sensors, ultrasound transducer array and two sets of pulse waveform sensor, each respectively according to an embodiment of the subject invention. From top to bottom, the names of the provided layers are skin friendly contact layer (410), two sets of pulse waveform sensor (420), ultrasound transducer common ground electrode layer (430), cavity (440), a copper cube (450), ultrasound transducer array (460), capsulation material (ecoflex) (470), ultrasound transducer stimulating electrode layer (480), skin friendly contact layer (490), according to an embodiment of the subject invention.

FIG. 7 illustrates two sets of pulse waveform sensors (500) according to an embodiment of the subject invention, showing overall device structure of a PVDF pressure sensor (420), including its serpentine electrode structure and cavity (440), according to an embodiment of the subject invention.

FIG. 8 illustrates the ultrasound array system (600) according to an embodiment of the subject invention, showing overall device structure of ultrasound transducer common ground electrode layer (430), a copper cube (450), ultrasound transducer array (460), ultrasound transducer stimulating electrode layer (480), according to an embodiment of the subject invention.

FIG. 9 illustrates the fabrication process of the ultrasound array system (600) according to an embodiment of the subject invention, the process comprising (710) spin coating PDMS; (720) defining electrode pattern; (730) removing excess electrode material; (740) peeling off electrode; (750) transferring bottom electrode onto Ecoflex substrate; (760) welding 1-3 composite and Cu cube; (770) aligning top electrode with the 1-3 composite; (780) bonding the top electrode and removing PDMS/glass substrate, according to an embodiment of the subject invention.

FIG. 10 illustrates the fabrication process of the pulse waveform sensors (500) and integration of dual-modality device (400) according to an embodiment of the subject invention, the process comprising (810) spin coating PDMS; (820) defining PWV sensor pattern; (830) removing excess PVDF material; (840) peeling off PWV sensor; (850) transferring PWV sensor on Ecoflex substrate; (860) bonding PDMS cavity on PWV sensor; (870) aligning ultrasound sensor with the PWV sensor; (880) pouring Ecoflex and removing the glass slides after the Ecoflex cures, according to an embodiment of the subject invention.

FIG. 11 illustrates a finite simulation model established on ANSYS software to qualitatively show the comparation of signal sensitivity between a single PVDF pressure sensor and PVDF pressure sensor with cavity made by high stiffness silicon rubber. By applying a force of 1 mN to the center point of the pressure sensor's front end, it can be observed that the cavity-less pressure sensor generated a strain of 0.59 μm and a strain of 7.08με, while the pressure sensor with a cavity generated a strain of 80.06 μm and a strain of 185.36με. Since the PVDF material of the pressure sensor is piezoelectric, the magnitude of the voltage generated by the piezoelectric material when subjected to force depends on its instantaneous deformation, according to an embodiment of the subject invention.

FIGS. 12A-12C illustrates the performance of the sensors, wherein FIG. 12A illustrates the anterior wall peak and posterior wall peak obtained by the ultrasound transducer during the measurement of blood vessel diameter, by calculating the time difference between these two peaks, the continuous variation of the vessel diameter over time can be determined as shown in FIG. 12B, and FIG. 12C illustrates the pulse waveform measured by two sets of pulse waveform sensors, according to an embodiment of the subject invention.

FIGS. 13A-13B illustrates the blood pressure assessment results of a single-modality device and algorithm as well as our dual-modality device and algorithm, wherein FIG. 13A illustrates BP waveforms obtained by single-modality device and algorithm and the error between the calculated BP waveform and the reference BP waveform, and FIG. 13B illustrates BP waveforms obtained by our dual-modality device and algorithm and the error between the calculated BP waveform and the reference BP waveform, according to an embodiment of the subject invention.

### DETAILED DISCLOSURE OF THE INVENTION

The embodiments of subject invention pertain to a dual-modality method and systems for measuring cardiovascular parameters and extracting blood pressure (BP)-related vascular characteristics.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present invention provides an accurate and effective dual-modality method for measuring cardiovascular parameters with BP-related vascular characteristic extraction, processing. The vascular characteristic extraction and processing method comprises the steps of:
1. Extracting the basic blood vessel parameters based on the signals measured from a dual-modality device.
2. Combining the basic vascular parameters to specify the individual vascular parameters.
3. Determining BP-related vascular characteristic parameters from the individual specific vascular parameters, which provides a comprehensive vascular characteristic expression of BP in the vessels underneath the body part being measured.

In one embodiment, the subject invention provides a dual-modality method which includes both a software design for a cuffless and calibration-free blood pressure (BP) method, as well as corresponding hardware designs for flexible and stretchable devices and systems.

In the software design part, the embodiments provide a cuffless and calibration-free blood pressure (BP) method that includes a BP-related vascular characterization extraction method and a pulse time decay compensation strategy.

The embodiments provide a BP-related vascular characterization extraction method that enables the extraction of comprehensive vascular conditions. The method is based on haemodynamic changes in individual dynamic vessel diameter, cross-sectional area and arterial stiffness to perform BP monitoring. These vascular characteristics can be directly extracted by the dual-modality device with ultrasound and pulse sensors. Based on this data, the method can determine different types of arterial stiffness parameters, including pulse wave velocity, pulse stiffening ratio, and beta stiffness index. By integrating detailed vascular analysis, the cuffless BP method offers a more comprehensive framework for interpreting the relationship between cardiovascular parameters and blood pressure. This approach surpasses the limitations of traditional cuff-based methods, which only provide a snapshot of BP without the supporting vascular context.

The embodiments also include a pulse time decay compensation strategy for blood pressure (BP) that allows the dual-modality device to accurately measure continuous BP. This time decay BP compensation strategy exploits the consistent trend between the time decay of BP and the pulse waveform extracted by the device's sensors. Specifically, the time decay BP compensation strategy takes advantage of the real-time pulse time decay combined with the individual dynamic blood vessel diameters measured by the device, allowing continuous BP-related time decay calculation and providing accurate BP trend tracking over time. Notably, the extraction of continuous and accurate pulse time decay features from the pulse waveforms heavily depends on the stability and reliability of the dual-modality device. By leveraging this time decay compensation strategy, the device can provide reliable, cuffless continuous BP monitoring across diverse populations and different measurement scenarios.

In the hardware design part, the embodiments provide a dual-modality device which includes ultrasound sensors and pulse sensors. The ultrasound sensor is configured to measure the geometric parameters of the measured blood vessel. The pulse sensors are configured to measure the proximal and distal pulse along the artery for further vascular hemodynamic parameter extraction.

Embodiments of the subject invention provide devices outperform the traditional technologies in terms of performance, accessibility, and safety.

Embodiments of the subject invention provide a design and fabrication protocol of a dual-modality device with ultrasound sensor array and a pressure sensor array, advantageously configured, and adapted to (a) measure real-time variations in blood vessel diameter using the ultrasound sensor, and (b) detect pulse wave velocity using the pressure sensor. In certain embodiments, a method for fabricating a flexible layered structure is provided to establish conformal contact with the skin. Embodiments of this device also provide a layered layout of the device and a multi-step layered fabrication protocol for realizing monolithic integration of the bimodal device. The entire device is stretchable and flexible, such that it can be attached to human skin for long-term blood pressure measurement.

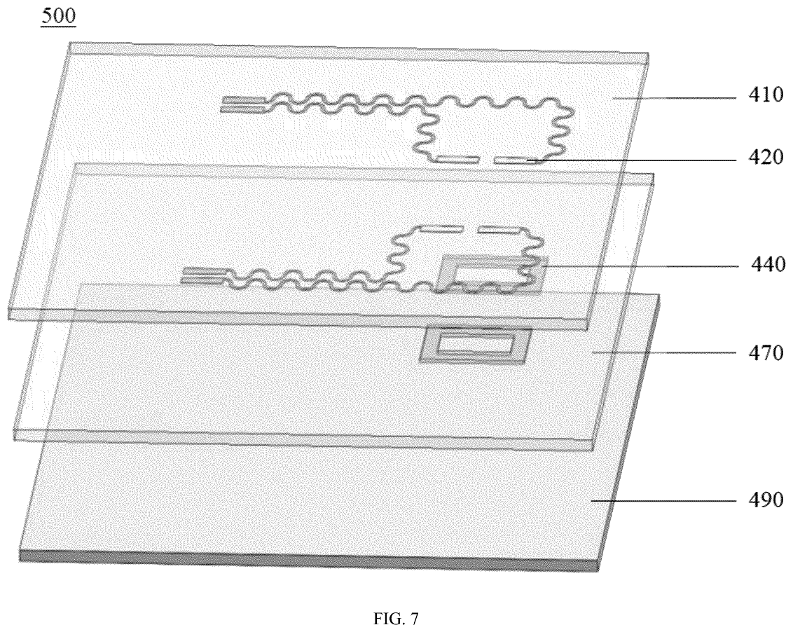

FIG. 6 illustrates an exemplary embodiment of the layout of the provided dual-modality device, where an ultrasound sensor array (for example, ultrasound transducer) layer (450) and two sets of pulse waveform sensors (PWS, 420) comprising a pair of thin conductive flexible electrodes are aligned in the layered concentric substrates. In certain embodiments, the device positioned on or in the skin friendly contact layer (410) can provide close contact with skin for long-term blood pressure measurement.

The pulse waveform sensors (420) are made by pressure sensors, including piezoelectric, piezoresistive, capacitive, and other type of sensors. Here, taking a polyvinylidene difluoride (PVDF), sensor as an example, polyvinylidene difluoride (PVDF) is a commonly used material in flexible pressure sensor, due to its excellent piezoelectric performance and flexibility. The sensing part of the PVDF film is cut into a rectangular shape to serve as a deformable beam, and the other part is cut into a serpentine structure, allowing it to withstand bending and tensile deformations. The PWS is placed on a thin silicon rubber film with low stiffness (Ecoflex 00-30, Young's modulus: ˜60 kPa), and a cavity (440) made by high stiffness silicon rubber (PDMS, Young's modulus: ˜1 MPa) is located on the rectangular PVDF beam to form a piezo cantilever, which can greatly improve the force sensitivity of the PWS. The ultrasound sensor array (460) includes 2×4 transduces with resonant frequency in the range of 1 MHz-10 M Hz. The transducer is made by 1-3 composites which have superior electromechanical coupling coefficients (thickness mode) that convert the majority of electrical energy to vibrational energy. In addition, the 1-3 composite possesses better acoustic coupling with the soft biological tissue than isotropic piezoelectric materials. Moreover, any other piezoelectric materials including but not limited to Lead zirconate titanate (PZT), Pb(Mg1/3Nb2/3)O3-PbTiO3 (PMN-PT) ceramics, and other suitable materials as known in the art, as well as CMUT, PMCT, can also be adopted as the ultrasound transducer. A conductive cube (450) is utilized as the vertical interconnect access to convert the upper and bottom electrodes to the same plane for the ease of electrical bonding. The total device is encapsulated by a low stiffness silicon rubber (for example, Ecoflex 00-30), such that it is flexible and stretchable enough to be adhered to the skin.

To ensure the flexibility and stretchability of the device, an “island-bridge” layout with the serpentine electrode (480) is exploited for ultrasound sensor array. The footprint is designed than the cross-section area transducer to balance practical bonding robustness and impedance matching. The serpentine electrodes can be made from metal, bilayer stacking of polymer and metal, conductive polymer, or other conductive materials. The island-bridge structure includes isolated electrode islands connected by bridge-like serpentine traces. This layout allows for improved flexibility and stretchability compared to traditional solid electrode designs. By distributing the electrode into smaller islands, the stress concentration is reduced, enabling the device to withstand higher levels of mechanical strain without compromising its performance. The serpentine electrode configuration further enhances the device's mechanical and electrical properties. The serpentine pattern allows for increased elongation and deformation, ensuring the electrode can stretch and bend along with the device without causing damage or signal loss. This design also helps to distribute stress evenly across the electrode to avoid localized failure points.

Furthermore, in order to ensure the overall stretchability of the device and protect the sensors, a low stiffness silicon rubber (for example, Ecoflex 00-30) is used as the encapsulation material (470). Low stiffness silicon rubber (for example, Ecoflex 00-30) is a highly commendable choice for its exceptional properties and performance. It is a flexible and stretchable material that exhibits excellent elasticity, durability, and biocompatibility. Its unique composition provides a high degree of protection to the embedded sensors, shielding them from external factors such as moisture, dust, and mechanical stress. Moreover, low stiffness silicon rubber (for example, Ecoflex 00-30) maintains its mechanical integrity even under repeated stretching and bending, making it an ideal choice for applications requiring long-term reliability. Additionally, its biocompatibility ensures compatibility with the human skin, reducing the risk of irritation or adverse reactions. With its remarkable combination of flexibility, durability, and biocompatibility, Ecoflex excels as a superior encapsulation material for ensuring the functionality and longevity of the device.

Aside from the preferred embodiment or embodiments disclosed below, the subject invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer. Identical elements in various figures are designated with the same reference numerals.

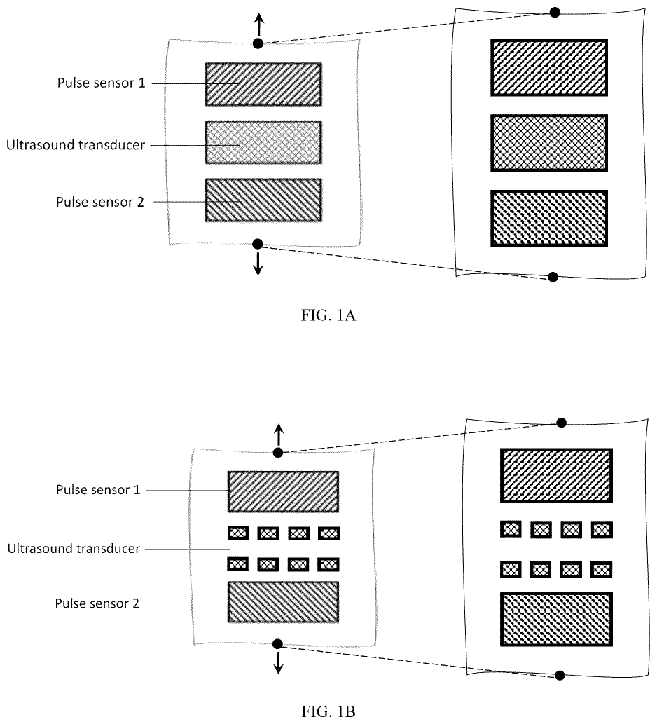

FIG. 1A shows one embodiment of dual-modality device patch with a central ultrasound sensor and a two-end pulse sensor. The central ultrasound sensor and a two-end pulse sensor are small in size or flexible so as to improve the conformity to the skin and comfortableness for the user. As the device measures echo and pulse signals, it can be placed on various body surface like carotid, wrist, leg, face, or other suitable locations. In one embodiment, the central ultrasound sensor is a flexible and stretchable ultrasound transducer component. The two-end pulse sensor can be a flexible and stretchable pressure or plethysmography-based components. The patch should be closely packed thus ensuring they attach to the same tissue/skin. In one non-limiting example, to acquire the echo and pulse signals in this sensor arrangement, ultrasound and pulse sensors are turned on simultaneously, then all these signals will be collected by a DAC module. In addition, the resonant frequency of ultrasound sensor is appropriately set to achieve desirable signal-to-noise ratios.

Referring to FIG. 1B, the central ultrasound sensor can also include multiple sub-elements with high selectivity and resolution to measure the echo signals from blood vessels. In one non-limiting example, the multiple sub-element ultrasound sensor can be switched by a multiplexer. As a result, the ultrasound sensor can generate the transmit and detect the echo signals corresponding to target measured blood vessels.

Referring to FIG. 1C, the two-end pulse sensor can also include multiple sub-elements with high selectivity and resolution to measure the pulse or volume signals from blood vessels. In one non-limiting example, the multiple sub-elements two-end pulse sensor can be switched by a multiplexer. As a result, the two-end pulse sensor can generate the map of pulse or volume signals corresponding to target measured blood vessels.

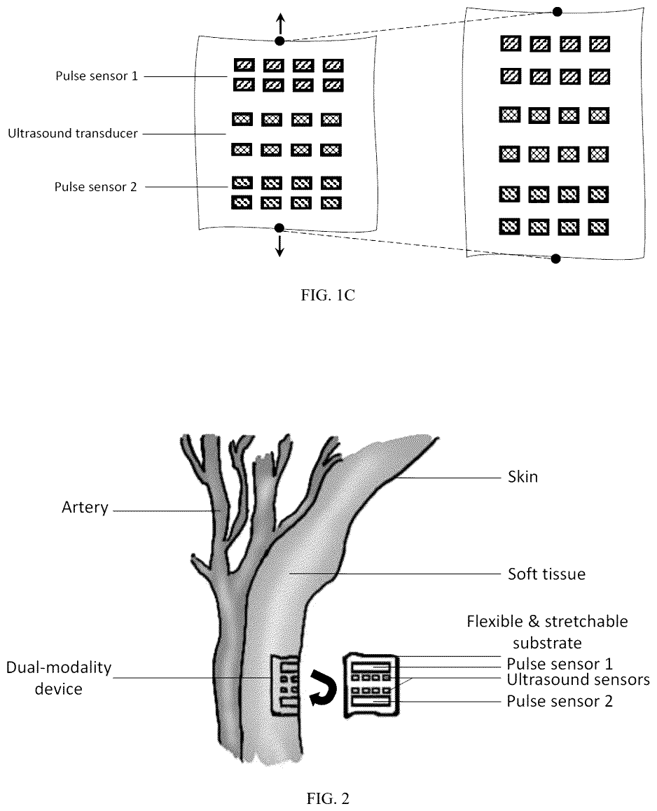

In FIG. 2, the physiological structure and dual-modality device interaction with the tissue are shown. The dual-modality device is designed to monitor the physiological structures located beneath the skin at the application site, typically on the carotid, arm, or wrist. This includes the outer layer of the skin (epidermis), the underlying arteries (such as the radial or brachial arteries), the walls of these blood vessels, and the microvascular network within the subcutaneous tissue. As the heart pumps, the arteries expand and contract with the pulsatile flow of blood, causing subtle changes in the properties of the vessel walls and the surrounding tissues.

The wearable ultrasound patch contains miniaturized ultrasound transducers emitting high-frequency sound waves, which penetrate through the skin layers into these underlying physiological structures. As the blood moves through the arteries, the movement causes changes in the reflection of the ultrasound waves. By analyzing the variations in the reflected ultrasound signal over time, the patch can accurately track key cardiovascular parameters, such as pulse rate, and changes in arterial diameter. In addition to the ultrasound sensors, the patch also incorporates pulse sensors that directly detect the pulsatile blood flow and pulse wave velocity, in the underlying vessels. This dual-modality approach, combining both ultrasound-based and pulse-based monitoring, enables the patch to provide a comprehensive, personalized profile of the user's unique cardiovascular and tissue health characteristics.

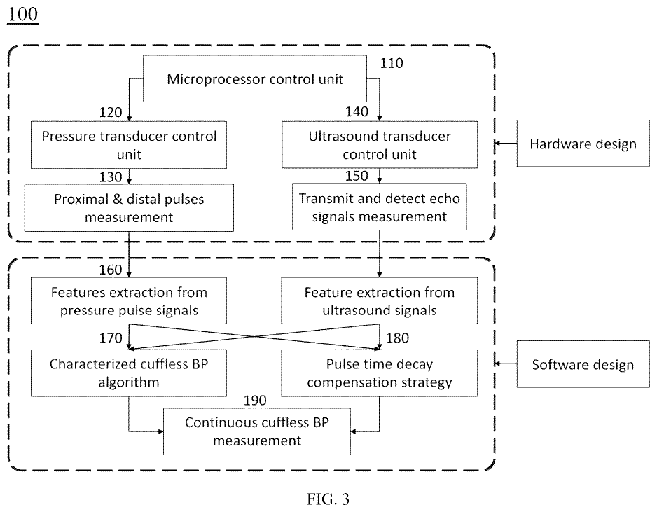

The whole framework of the dual-modality device for blood pressure measurement is shown in FIG. 3, including the hardware and software design. In the hardware design, the microprocessor control unit (110) is configured for coordinating the synchronized data collection from the pressure and ultrasonic sensors. The pressure transducer control unit (120) measures the proximal and distal pulse waveforms (130), while the ultrasonic control unit (140) is configured for transmitting and detecting the echo signals (150) of the body's blood vessel diameters. In the software design, features (160) are extracted from the pulse waveforms and echo signals. The extracted features (160) are then utilized for continuous cuffless BP measurement (190) via the BP-related vascular characterization extraction method of cuffless BP measurement (170) and the pulse time decay BP compensation strategy (180).

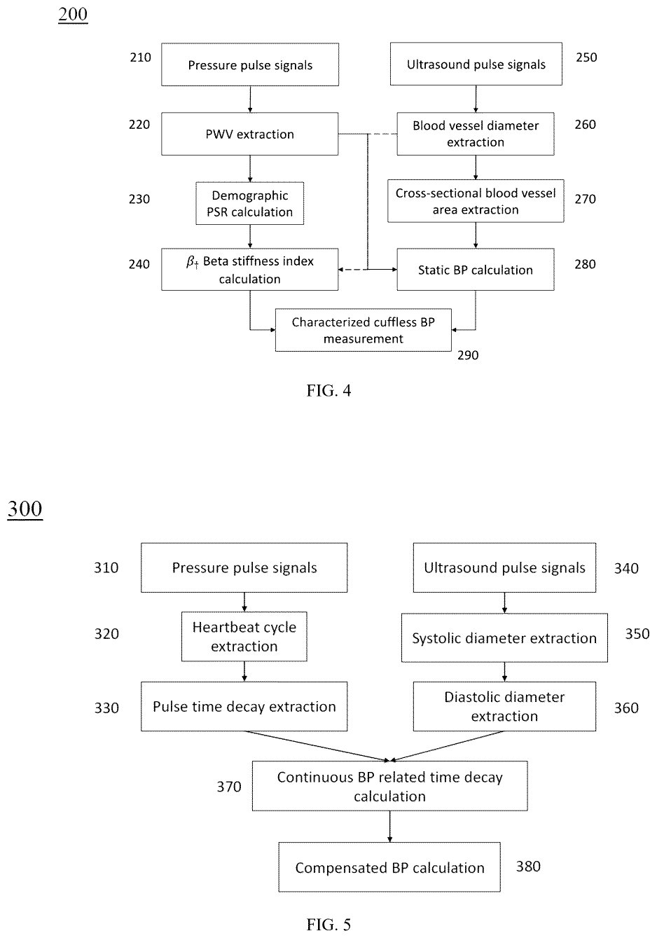

In FIG. 4, the dual-modality method of BP-related vascular characterization extraction method for BP measurement involves assessment of arterial dimensions by ultrasound echo technique, and local PWV values by the two-point method from time-shifted blood pulse. The method is based on the pulse wave velocity (PWV) (220) extracted from the pressure pulse signals (210), as well as the blood vessel diameters (260) and cross-sectional blood vessel area (270) extracted from the ultrasound pulse signals (250). These inputs enable the calculation of the pulse stiffening ratio (PSR) (230) and beta stiffness index (240). By combining the beta stiffness index (240) and static blood pressure (280), the method can perform the characterized beta stiffness induced cuffless blood pressure measurements (290).

Consider a general relationship of pressure and cross-sectional area in an arterial segment [4], which can be described by

$$P = θ + {Y}^{{e}^{β \cdot A}} \qquad ( 1 )$$

where β and γ are pressure-independent constants. θ is related to intrinsic properties of an artery to remain patent at very low pressures and γ is a scaling factor for the y-intercept. Area is the cross-sectional area of the arterial segment. β is the beta stiffness, which is expressed as

$$β = \frac{ln [ ( S B P - θ ) / ( D B P - θ ) ]}{Δ A} \qquad ( 2 )$$

where SBP is systolic blood pressure, DBP is diastolic blood pressure, and ΔA is arterial cross-sectional area difference between systolic and diastolic phase.

Then the BP components can be expressed as:

$$S B P = P S R \cdot D B P + θ ( 1 - P S R ) \qquad ( 3 )$$

where PSR is pulsatile stiffening ratio.

$$P S R = {e}^{β \cdot Δ A} \qquad ( 4 )$$

Accordingly, the beta stiffness can be derived:

$$β = ln ( P S R ) / Δ A \qquad ( 5 )$$

where PSR is a constant, ΔA is the pulse area and can be measured by ultrasound sensor.

Since PSR is equivalent to ambulatory arterial stiffness index (AASI) and AASI is positively proportional to pulse wave velocity [5]. Hence,

$$β = ln ( 1 - 1 / ( a \cdot P W V + b ) ) / Δ A \qquad ( 6 )$$

where a and b are constants and associate with the relation of PSR and PWV in population characteristic.

On the other hand, based on Equation (1), the variation of area is expressed as the variation of the BP:

$$\frac{dP}{dA} = ( P - θ ) \cdot β \qquad ( 7 )$$

Furthermore, the relation between PWV and BP is expressed as follows [6]:

$$P W {V}^{2} = \frac{A}{ρ} \frac{dP}{dA} \qquad ( 8 )$$

where ρ is the blood density.

Thus, BP can be expressed as the combination of vascular characteristic parameters:

$$P = \frac{P W {V}^{2}}{A \cdot β / ρ} + θ \qquad ( 9 )$$

By substituting beta stiffness in Equation (9), the beta stiffness induced BP (BP_{b}) is expressed as follows:

$$B {P}_{b} = \frac{P W {V}^{2}}{ln ( 1 - 1 / ( a \cdot P W V + b ) ) / ρ \cdot A / ( A - {A}_{d} )} + θ \qquad ( 10 )$$

where PWV is the pulse wave velocity; A is the cross-sectional area of arteries; Ad is the initial area; and a, b and θ are constants.

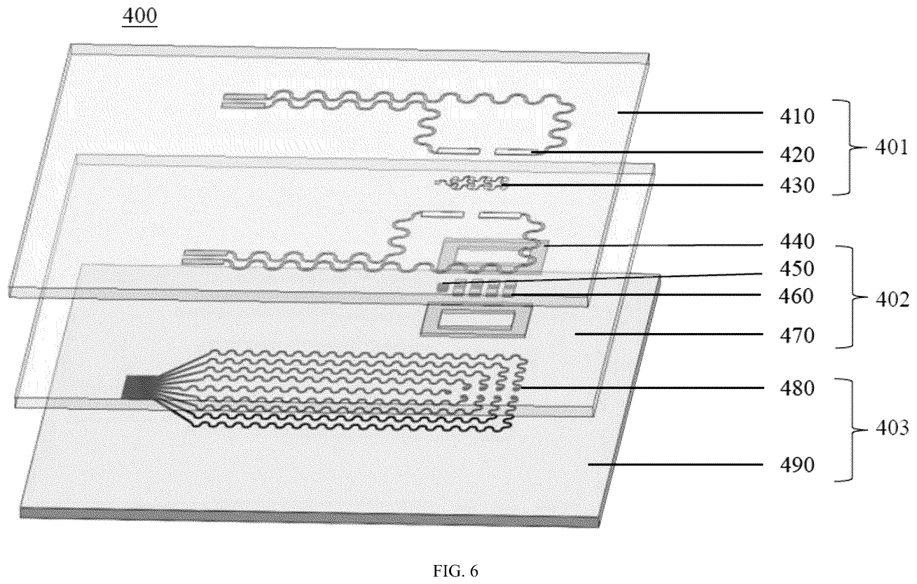

FIG. 5 illustrates the pulse time decay BP compensation strategy (180) that incorporates multiple features extracted from both the pressure pulse signals (310) and ultrasound pulse signals (340). The extracted features include the heartbeat cycle (320), pulse time decay (330), systolic diameter (360), and diastolic diameter (370). Based on these extracted features, the continuous BP-related time decay (340) can be calculated and used for the continuous BP monitoring (380).

The BP pulse time decay calculation is expressed as follows:

$${τ}_{BP} = \frac{{T}_{c}}{{β}_{†} [ \frac{{D}_{s}^{*}}{{D}_{d}} \cdot ( 1 - {e}^{- \frac{{T}_{c}}{{τ}_{D}}} ) ]} \qquad ( 11 )$$

where the T_{c} is the heartbeat cycle; τ_{BP} is the pulse time decay of BP; τ_{D} is the pulse time decay of the measured pulse waveforms from sensors; Dₛ* is the systolic blood vessel diameter; and D_{d} is the diastolic blood vessel diameter. The τ_{BP} captures the trend of BP fluctuation that compensates the characterized cuffless BP in equation (10). Finally, the continuous BP (BP_{c}) can be expressed as follows:

$$B {P}_{c} = c \cdot {τ}_{BP} / {τ}_{{BP}_{0}} + d + B {P}_{b} \qquad ( 12 )$$

where c and d are preset constants, and τ_{BP}_{₀} is the initial time decay corresponding to the characterized cuffless BP measurement.

In certain embodiments of the subject invention, the device of the subject invention can be divided into two parts: (1) ultrasound transducer array system, and (2) two set of pulse waveform sensors, such as pressure sensor using PVDF. The device can be assembled in an orientation such that the top, or last surface to be assembled, can be the first surface to contact the skin. The fabricated layers of the provided device from top to bottom can comprise:
Layer 1 (401): skin friendly contact layer (410), two sets of pulse waveform sensor (420), ultrasound transducer common ground electrode layer (430);
Layer 2 (402): cavity (440), conductive cube (450), ultrasound transducer array (460), capsulation material (for example, Ecoflex) (470); and
Layer 3 (403): ultrasound transducer stimulating electrode layer (480), skin friendly contact layer (490).

In certain embodiments, the dual-modality device mainly includes two sensing unit as shown in FIG. 6. The pulse waveform sensing unit comprises skin friendly contact layer (410), two sets of pulse waveform sensor (420), cavity (440), capsulation material (for example, Ecoflex) (470) and skin friendly contact layer (490). The ultrasound sensing unit comprises skin friendly contact layer (410), ultrasound transducer common ground electrode layer (430), conductive cube (450), ultrasound transducer array (460), ultrasound transducer stimulating electrode layer (480), and skin friendly contact layer (490).

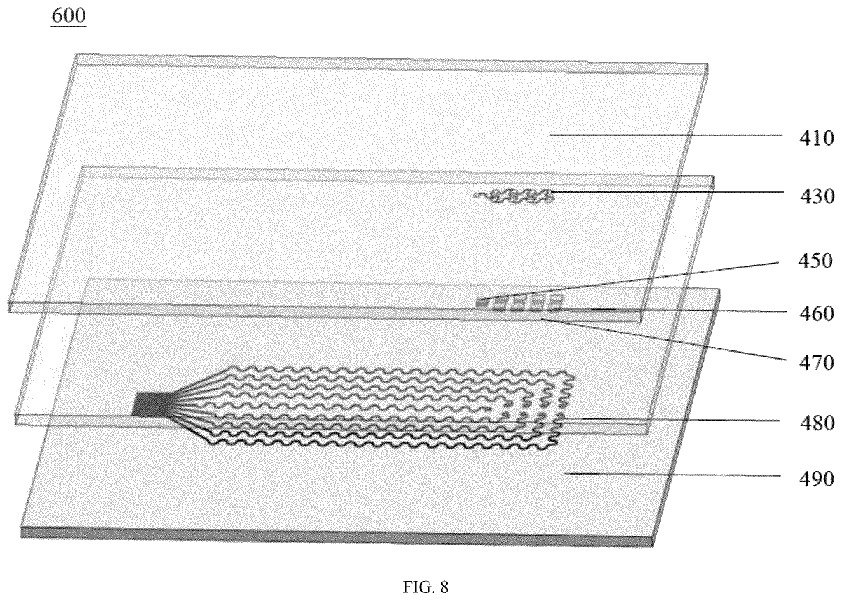

A pulse waveform sensing unit is shown in FIG. 7, which is used for local pulse wave velocity (PWV) measurement. The pulse waveform sensing unit comprises skin friendly contact layer (410), two sets of pulse waveform sensor (420), cavity (440), capsulation material (Ecoflex) (470) and skin friendly contact layer (490). PWV is a measure of arterial stiffness, or the rate at which pressure waves move down the vessel. As blood flows through the vessels of the circulatory system, it moves out of the left ventricle and into and aorta where it is then pushed through the rest of the circulatory system. The velocity of this movement gives a measurement of arterial compliance. Local PWV begins to garner increasing levels of interest because of the superiority of its distinctive features over conventional regional PWV. Recent studies have demonstrated that the local PWV is precisely related to the vascular characteristics, biomechanical information, transmural BP level, and pathophysiology of the artery under investigation. Local PWV is clinically significant and is being used as an indicator of the vascular damage and is a potential risk factor of coronary heart disease and stroke. Emerging evidence from various clinical studies has now established that the local PWV measured from the carotid artery is a robust surrogate marker of various cardiovascular diseases. The pulse waveform sensor can be made from piezoelectric, piezoresistive, capacitive, and other type of sensors.

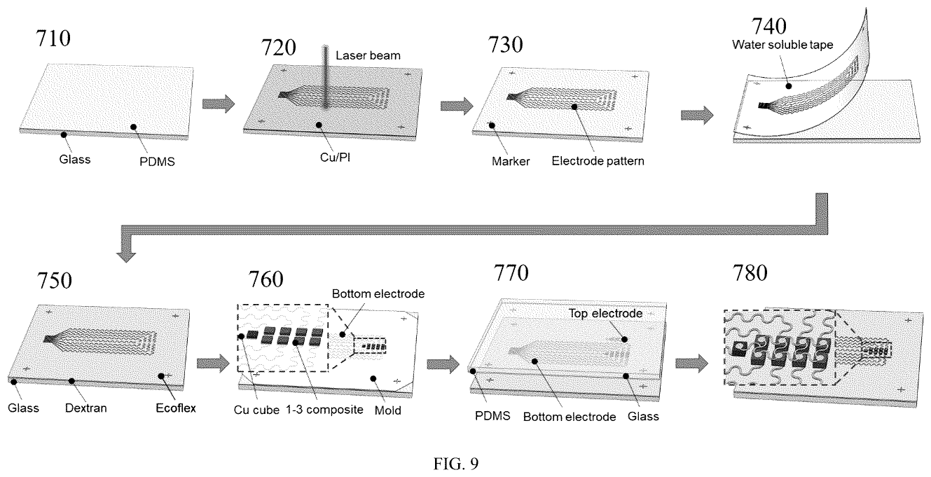

FIG. 8 presents the ultrasound sensing unit, which can continuously monitor diameter change of artery. The ultrasound sensing unit comprises skin friendly contact layer (410), ultrasound transducer common ground electrode layer (430), conductive cube (450), ultrasound transducer array (460), ultrasound transducer stimulating electrode layer (480), and skin friendly contact layer (490). Continuous monitoring of the diameter changes can evaluate the overall health and function of the vascular system, detecting conditions such as atherosclerosis, arterial stiffness, or vascular inflammation at an early stage. Furthermore, blood vessel diameter variations serve as important indicators of cardiovascular risk factors, facilitating risk assessment and guiding preventive measures. Additionally, tracking these changes over time allows for the evaluation of treatment efficacy, ensuring optimal patient care. The conventional ultrasound devices are rigid and bulky, too hard to support real time monitoring, and do not have a solid interfacial contact and therefore good coupling with irregular nonplanar surfaces [7].

Herein, an ultrasound sensing unit that can be easily attach to human skin and measure the diameter of the vessel is provided, making it possible for real time and long-term monitoring the signals of cardiovascular. The sensors can be made from 1-3 composite or other piezoelectric material, CMUT, PMCT can also be adopted, which has superior electromechanical coupling coefficients (thickness mode) that convert the majority of electrical energy to vibrational energy. In addition, the 1-3 composite possesses better acoustic coupling with the soft biological tissue than isotropic piezoelectric materials.

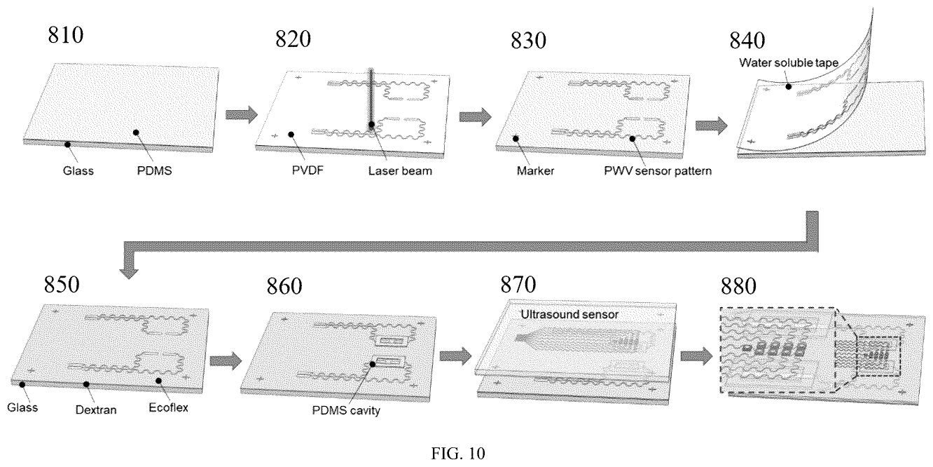

FIG. 9 presents a general fabrication process of the ultrasound array system (600) according to an embodiment of the subject invention, which comprises following steps:
(710) spin coating PDMS on a glass slide;
(720) laminating Cu/PI foil on PDMS and defining electrode pattern by a laser cutter;
(730) removing excess electrode materials;
(740) peeling off electrode by a water soluble tape;
(750) transferring bottom electrode on Ecoflex substrate spin-coated on a dextran-decorated glass slide;
(760) welding 1-3 composite and Cu cube on a bottom electrode;
(770) aligning top electrode with the 1-3 composites;
(780) bonding the top electrode and remove PDMS/glass substrate.

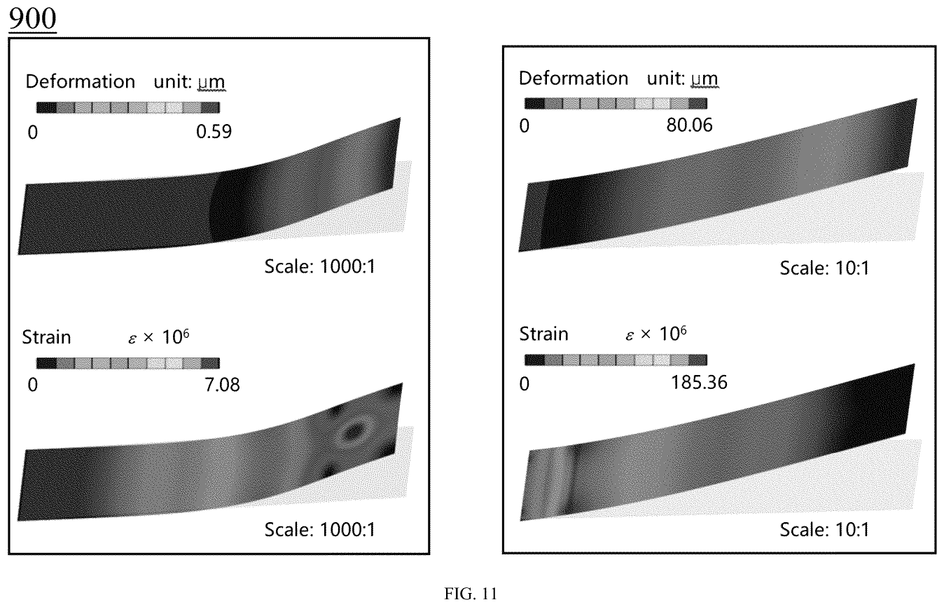

FIG. 10 presents a general fabrication process of the pulse waveform sensors (500) and integration of dual-modality device (400) according to an embodiment of the subject invention, which comprises following steps:
(810) spin coating PDMS on a glass slide;
(820) laminating PVDF film on PDMS, and define PWV sensor pattern by laser cutter;
(830) removing excess PVDF materials;
(840) peeling off PWV sensor by a water soluble tape;
(850) transferring Transfer PWV sensor on an Ecoflex substrate spin-coated on a dextran-decorated glass slide;
(860) bonding PDMS cavity on a PWV sensor;
(870) aligning ultrasound sensor with the PWV sensor;
(880) pouring Ecoflex and removing the glass slides after the Ecoflex cures.

It has been proven that sensor devices combining ultrasound and pressure sensors are effective for blood pressure measurement. Common measurement methods include Mercury sphygmomanometer, pulse wave analysis, Digital blood pressure monitor, and other methods known in art.

Embodiments of the subject invention are not only mechanically flexible (for example, with the ability of bending to a radius as small as 10 mm), allowing for good contact with the skin, but also capable of real-time monitoring of continuous blood pressure waveform. The system of the subject invention performs completed blood pressure and the demographic-based arterial stiffness parameters measurement, such as beta stiffness, pulsatile stiffening ratio, and ambulatory arterial stiffness index. The system of the subject invention provides a slippage-free continuous BP tracking technique that gets rid of the skin interface issue of the patch-based sensor faced with sensor slippage, position deviation, and blood vessel drift during BP measurements.

Moreover, the embodiments of the subject invention generate several innovations that are critical for conducting a precise and convenient measurement. For example, the invention introduces a dual array approach comprising an ultrasonic sensor array and a pressure sensor array. This setup significantly enhances the measurement position tolerance, effectively meeting the practical requirements of blood pressure assessment.

In an embodiment of the subject invention, the device integrates an ultrasound sensor array for blood vessel diameter measurements alongside a pressure sensor array for local pulse wave velocity (PWV) assessments. The setup enables the simultaneous measurement of two blood vessel signals in the same pulse cycle, ensuring enhanced accuracy in blood pressure readings.

Embodiments of the subject invention address the technical problem of controlling the application of flexible device to provide continuous and precise measurement of blood pressure being rigid and bulky, needing excessive human processing, not being suitable for wearable applications, and complex equipment.

This problem is addressed by providing a dual-modality device with ultrasound transducer array and pulse waveform sensor and algorithm model in a compact, wearable, and flexible device. The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

### Materials and Methods

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

The following examples represent select prototype flexible blood pressure monitoring devices with different sensors and blood pressure algorithm models, fabricated and tested by the inventors according to embodiments of the subject invention. The fabrication process and materials used have been developed and optimized. Both theoretical modeling and experimental results have confirmed the superior efficacy and practicality of the provided dual-mode blood pressure measurement system.

Following are examples that illustrate procedures for practicing the invention. These examples should not be construed as limiting. All percentages are by weight and all solvent mixture proportions are by volume unless otherwise noted.

### Example 1

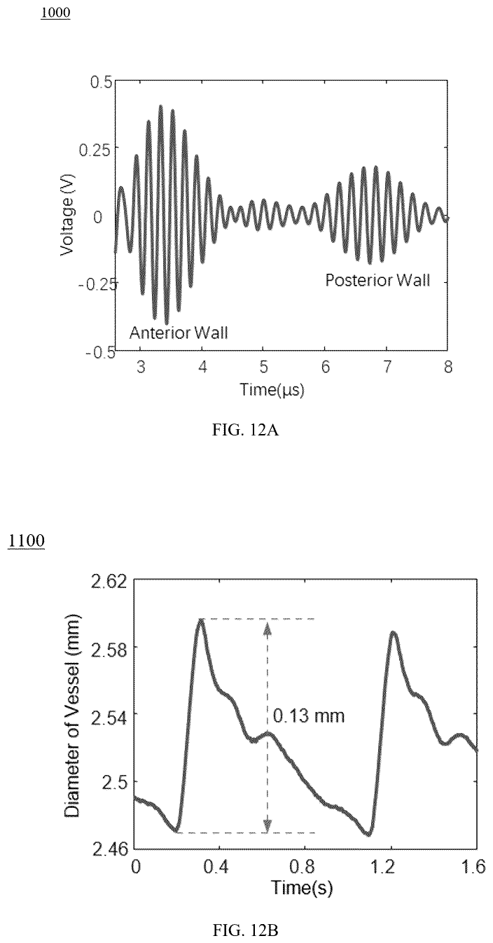

A finite element simulation model is established on ANSYS software to qualitatively illustrate the comparation of signal sensitivity between a single PVDF pressure sensor and PVDF pressure sensor with cavity made by high stiffness silicon rubber (PDMS, Young's modulus: ˜1 MPa), as shown in FIG. 11. By applying a force of 1 mN to the central point of the pressure sensor's front end, it can be observed that the cavity-less pressure sensor generated a strain of 0.59 μm and a strain of 7.08με, while the pressure sensor with a cavity generated a strain of 80.06 μm and a strain of 185.36με. Since the PVDF material of the pressure sensor is piezoelectric, the magnitude of the voltage generated by the piezoelectric material when subjected to force depends on its instantaneous deformation. Therefore, these results indicate that the pressure sensor with a cavity structure performs significantly better than the cavity-less pressure sensor, leading to important improvements of the accuracy of measuring pulse waveforms. The cavity structure in the pressure sensor improves its performance by allowing for greater deformation of the sensing element, resulting in increased sensitivity and accuracy in pressure measurements. Additionally, the cavity helps to shield the sensing element from external disturbances, ensuring more reliable and accurate pressure readings. Overall, the cavity structure enhances the sensor's sensitivity, amplifies the applied force, and protects against external influences, leading to improved performance in pressure measurements.

### Example 2

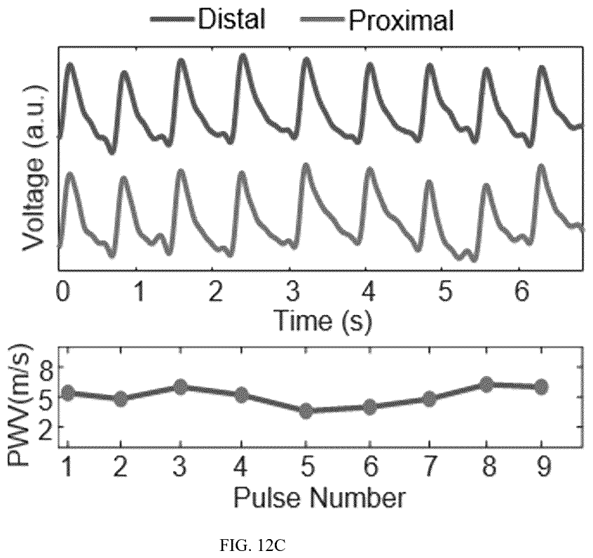

To validate the effectiveness of the device, further testing is conducted on the pulse waveform sensor and ultrasound transducer array. By placing the device on the wrist, we performed measurements on the arterial vessels in this region. FIG. 12A illustrates the anterior wall peak and posterior wall peak obtained by the ultrasound transducer during the measurement of blood vessel diameter. By calculating the time difference between these two peaks, the continuous variation of the vessel diameter over time is determined as shown in FIG. 12B. In addition, as shown in FIG. 12C, two sets of pulse waveform sensors are utilized to measure the time difference in the signals generated by the pulse wave at different positions, as well as the distance between the two sensors, allowing obtaining the local pulse wave velocity (local PWV). These signals demonstrate the outstanding performance of our integrated device. The accurate measurements of blood vessel diameter change and precise assessment of local PWV demonstrate the device's reliability and effectiveness. The findings highlight the devices potential for clinical applications.

### Example 3

To assess the merits of the device and method, a continuous blood pressure evaluation is conducted. Throughout the test, all subjects maintained a seated position, with results obtained from a commercial continuous blood pressure system (BIOPAC, MP160) utilized as reference values.

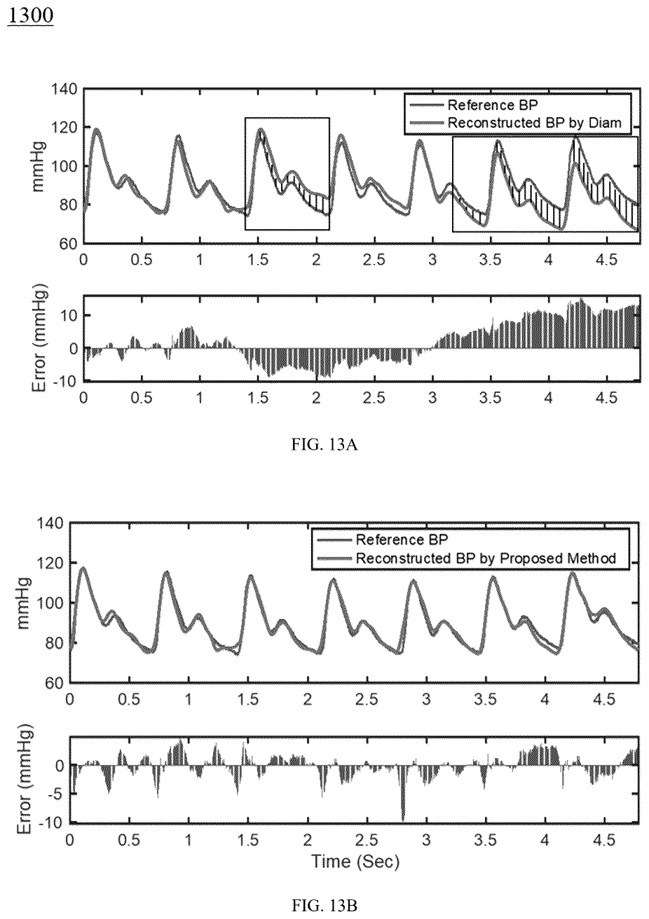

FIGS. 13A and 13B present the blood pressure assessment outcomes achieved using both a single-modality device and algorithm, as well as our dual-modality device and algorithm. Specifically, FIG. 13A showcases the BP waveforms and the error between the calculated BP values and the reference BP values derived from the single-modality device and algorithm. On the other hand, FIG. 13B displays the BP waveforms and the error between the calculated BP waveforms and the reference BP waveforms obtained from our dual-modality device and method.

The results indicate a high level of consistency between the BP value waveforms calculated by both methods and the reference values across two pulse cycles. However, beyond this point, the BP waveform derived from the single-modality device and method tends to deviate from the reference BP waveform, leading to measurement inaccuracies. In contrast, the BP waveform derived from the dual-modality device and algorithm remains aligned with the reference BP waveform even after two cycles, underscoring the effectiveness of the approach outlined in this patent for continuous blood pressure monitoring.

### EXEMPLARY EMBODIMENTS

Embodiment 1. A dual-modality method for measuring cardiovascular parameters and extracting blood pressure (BP)-related vascular characteristics, the method comprising:
extracting basic blood vessel parameters from signals measured by a dual-modality device;
combining the basic blood vessel parameters to determine individual vascular parameters; and
determining comprehensive BP-related vascular characteristic parameters from the individual vascular parameters, for providing a vascular profile related to blood pressure.
Embodiment 2. The method of embodiment 1, wherein the blood vessel parameters include a dynamic vessel diameter, a cross-sectional area of vessels, and arterial stiffness.
Embodiment 3. The method of any preceding embodiment, wherein the individual vascular parameters include pulse wave velocities, pulse stiffening ratios, and dynamic beta stiffness indexes.
Embodiment 4. The method of any preceding embodiment, wherein the BP-related vascular characteristic parameters are configured to enable individual calibration-free BP measurement and continuous BP tracking.
Embodiment 5. The method of any preceding embodiment, wherein the dual-modality device includes both ultrasound and pulse sensors to enable stable and reliable extraction of vascular data to support a cuffless BP method.
Embodiment 6. The method of any preceding embodiment, further comprising cuffless and calibration-free blood pressure (BP) steps including:
BP-related vascular characterization extraction steps; and
pulse time decay compensation steps.
Embodiment 7. The method of any preceding embodiment, wherein each of the ultrasound sensors includes a plurality of elements.
Embodiment 8. The method of any preceding embodiment, wherein each of the ultrasound sensors includes one element or a plurality of elements and the pulse sensors include a pressure-based sensor, and/or a plethysmography-based sensor.
Embodiment 9. The method of any preceding embodiment, wherein the BP-related vascular characterization extraction steps extract features from the pulse signals and ultrasound signals by the dual-modality device.
Embodiment 10. The method of any preceding embodiment, wherein the BP-related vascular characterization extraction steps enable the characteristic cuffless BP measurement based on the pulse wave velocities, blood vessel diameters, and cross-sectional blood vessel areas for individual static cuffless BP measurement.
Embodiment 11. The method of any preceding embodiment, wherein the BP-related vascular characterization extraction steps enable different types of arterial stiffness parameters calculation based on the pulse wave velocities, the pulse stiffening ratios, and the beta stiffness indexes.
Embodiment 12. The method of any preceding embodiment, wherein the BP-related vascular characterization extraction steps enable accurate BP monitoring by combing the static cuffless BP measurement with the dynamic beta stiffness.
Embodiment 13. The method of any preceding embodiment, wherein the pulse time decay compensation steps are based on a consistent trend between pulse waveform time decay and BP time decay and are configured by combining real-time pulse time decay and individual dynamic blood vessel diameters to perform continuous and accurate BP trend tracking.
Embodiment 14. The method of any preceding embodiment, wherein the pulse time decay BP compensation steps are configured to extract features from the pulse signals and ultrasound signals.
Embodiment 15. The method of any preceding embodiment, wherein the pulse time decay BP compensation steps are configured to calculate the time decay of BP based on the pulse waveforms.
Embodiment 16. The method of any preceding embodiment, wherein the pulse time decay BP compensation steps are configured to calculate the compensated BP based on the time decay of BP.
Embodiment 17. The method of any preceding embodiment, wherein the pulse time decay BP compensation steps are configured to permit accurately continuous BP measurement by combining the compensated BP and the characteristic cuffless BP.
Embodiment 18. A flexible dual-modality system for continuous blood pressure monitoring, the system comprising:
a first layer (401) comprising a skin friendly contact layer (410), a pulse waveform sensor (420), and an ultrasound transducer common ground electrode layer (430), wherein each set of pulse waveform sensors comprises two pressure sensors;
a second layer (402) comprising a cavity (440), a copper cube (450), an ultrasound transducer array (460), a capsulation material (470); and
a third layer (403) comprising an ultrasound transducer stimulating electrode layer (480), and a skin friendly contact layer (490).
Embodiment 19. The dual-modality system according to any preceding embodiment, further comprising at least two sets of pulse waveform sensors, two cavities, eight pixels, two skin friendly contact layers, and two electrode layers.
Embodiment 20. The dual-modality system according to any preceding embodiment, wherein the skin friendly contact (410) layer comprises a silicone rubber, Ecoflex, PDMS (polydimethylsiloxane), or SEBS.
Embodiment 21. The dual-modality system according to any preceding embodiment, further comprising two electrodes.
Embodiment 22. The dual-modality system according to any preceding embodiment, wherein the two electrodes are made from at least one material selected from the group consisting of gold (Au), silver (Ag), copper (Cu), another conductive metal, a conductive polymer, and a conductive non-metal, non-polymer material.
Embodiment 23. The dual-modality system according to any preceding embodiment, wherein the two electrodes are formed to have a serpentine structure having stretchability.
Embodiment 24. The dual-modality system according to any preceding embodiment, wherein the ultrasound sensing unit comprises ultrasound transducers (460) and copper cube (450).
Embodiment 25. The dual-modality system according to any preceding embodiment, the ultrasound transducer is formed of piezoelectric ceramics with 1-3 composite structure for performance in d₃₃ direction to improve acoustic coupling and electromechanical conversion.
Embodiment 26. The dual-modality system according to any preceding embodiment, wherein the copper cube (150) has a same thickness as that of the ultrasound transducer, allowing smooth routing of common ground electrode towards a same plane as that of the stimulating electrode.
Embodiment 27. The dual-modality system according to any preceding embodiment, wherein the pulse waveform sensing unit comprises a pressure sensor and a cavity.
Embodiment 28. The dual-modality system according to any preceding embodiment, wherein the pressure sensor is made by organic flexible piezoelectric materials including polyvinylidene difluoride (PVDF) and has a serpentine structure to withstand bending and tensile deformation.
Embodiment 29. The dual-modality system according to any preceding embodiment, wherein the cavity is made by a high stiffness silicon rubber including PDMS, the cavity is located on the rectangular pressure sensor beam to form a piezo cantilever to improve force sensitivity of the pressure sensor.
Embodiment 30. The dual-modality system according to any preceding embodiment, wherein the entire system is encapsulated by a silicone rubber with modulus on par with that of human skin to provide a barrier to moisture for protecting the system from sweat corrosion, to allow conformation to different surfaces, and to endure twisting and stretching.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] Zhao, L., Liang, C., Huang, Y. et al. Emerging sensing and modeling technologies for wearable and cuffless blood pressure monitoring. npj Digit. Med. 6, 93 (2023).
[2] Sheng X U. “Stretchable Ultrasonic Transducer Devices.” U.S. Patent Application No. 2019/0328354.
[3] Nabeel Pilaparambil Mashood. “Multi-Modal Ultrasound Probe for Calibration-Free Cuff-Less Evaluation of Blood Pressure.” U.S. Patent Application No. 2021/0077057.
[4] B. Gavish and J. L. Izzo, Jr., “Arterial Stiffness: Going a Step Beyond,” Am J Hypertens, vol. 29, no. 11, pp. 1223-1233 Nov. 1, 2016, doi: 10.1093/ajh/hpw061.
[5] Y. Li et al., “Ambulatory Arterial Stiffness Index Derived From 24-Hour Ambulatory Blood Pressure Monitoring,” Hypertension, vol. 47, no. 3, pp. 359-364, 2006, doi: doi:10.1161/01.HYP.0000200695.34024.4c.
[6] Fung Y C (1997) Biomechanics: Circulation (Springer, New York).
[7] Lin, M., Hu, H., Zhou, S. et al. Soft wearable devices for deep-tissue sensing. Nat Rev Mater 7, 850-869 (2022).

We claim:

1. A dual-modality method for measuring cardiovascular parameters and extracting blood pressure (BP)-related vascular characteristics, the method comprising:
extracting basic blood vessel parameters from signals measured by a dual-modality device;
combining the basic blood vessel parameters to determine individual vascular parameters; and
determining comprehensive BP-related vascular characteristic parameters from the individual vascular parameters, for providing a vascular profile related to blood pressure.

2. The method of claim 1, wherein the blood vessel parameters include a dynamic vessel diameter, a cross-sectional area of vessels, and arterial stiffness.

3. The method of claim 1, wherein the individual vascular parameters include pulse wave velocities, pulse stiffening ratios, and dynamic beta stiffness indexes.

4. The method of claim 1, wherein the BP-related vascular characteristic parameters are configured to enable individual calibration-free BP measurement and continuous BP tracking.

5. The method of claim 1, wherein the dual-modality device includes both ultrasound and pulse sensors to enable stable and reliable extraction of vascular data to support a cuffless BP method.

6. The method of claim 4, further comprising cuffless and calibration-free blood pressure (BP) steps including:
BP-related vascular characterization extraction steps; and
pulse time decay compensation steps.

7. The method of claim 5, wherein each of the ultrasound sensors includes a plurality of elements.

8. The method of claim 5, wherein each of the ultrasound sensors includes one element or a plurality of elements and the pulse sensors include a pressure-based sensor, and/or a plethysmography-based sensor.

9. The method of claim 6, wherein the BP-related vascular characterization extraction steps extract features from the pulse signals and ultrasound signals by the dual-modality device.

10. The method of claim 6, wherein the BP-related vascular characterization extraction steps enable the characteristic cuffless BP measurement based on the pulse wave velocities, blood vessel diameters, and cross-sectional blood vessel areas for individual static cuffless BP measurement.

11. The method of claim 6, wherein the BP-related vascular characterization extraction steps enable different types of arterial stiffness parameters calculation based on the pulse wave velocities, the pulse stiffening ratios, and the beta stiffness indexes.

12. The method of claim 6, wherein the BP-related vascular characterization extraction steps enable accurate BP monitoring by combing the static cuffless BP measurement with the dynamic beta stiffness.

13. The method of claim 6, wherein the pulse time decay compensation steps are based on a consistent trend between pulse waveform time decay and BP time decay and are configured by combining real-time pulse time decay and individual dynamic blood vessel diameters to perform continuous and accurate BP trend tracking.

14. The method of claim 6, wherein the pulse time decay BP compensation steps are configured to extract features from the pulse signals and ultrasound signals.

15. The method of claim 6, wherein the pulse time decay BP compensation steps are configured to calculate the time decay of BP based on the pulse waveforms.

16. The method of claim 6, wherein the pulse time decay BP compensation steps are configured to calculate the compensated BP based on the time decay of BP.

17. The method of claim 6, wherein the pulse time decay BP compensation steps are configured to permit accurately continuous BP measurement by combining the compensated BP and the characteristic cuffless BP.

18. A flexible dual-modality system for continuous blood pressure monitoring, the system comprising:
a first layer (401) comprising a skin friendly contact layer (410), a pulse waveform sensor (420), and an ultrasound transducer common ground electrode layer (430), wherein each set of pulse waveform sensors comprises two pressure sensors;
a second layer (402) comprising a cavity (440), a copper cube (450), an ultrasound transducer array (460), a capsulation material (470); and
a third layer (403) comprising an ultrasound transducer stimulating electrode layer (480), and a skin friendly contact layer (490).

19. The dual-modality system according to claim 18, further comprising at least two sets of pulse waveform sensors, two cavities, eight pixels, two skin friendly contact layers, and two electrode layers.

20. The dual-modality system according to claim 18, wherein the skin friendly contact (410) layer comprises a silicone rubber, Ecoflex, PDMS (polydimethylsiloxane), or SEBS.

21. The dual-modality system according to claim 18, further comprising two electrodes.

22. The dual-modality system according to claim 21, wherein the two electrodes are made from at least one material selected from the group consisting of gold (Au), silver (Ag), copper (Cu), another conductive metal, a conductive polymer, and a conductive non-metal, non-polymer material.

23. The dual-modality system according to claim 21, wherein the two electrodes are formed to have a serpentine structure having stretchability.

24. The dual-modality system according to claim 19, wherein the ultrasound sensing unit comprises ultrasound transducers (460) and copper cube (450).

25. The dual-modality system according to claim 24, the ultrasound transducer is formed of piezoelectric ceramics with 1-3 composite structure for performance in d₃₃ direction to improve acoustic coupling and electromechanical conversion.

26. The dual-modality system according to claim 24, wherein the copper cube (150) has a same thickness as that of the ultrasound transducer, allowing smooth routing of common ground electrode towards a same plane as that of the stimulating electrode.

27. The dual-modality system according to claim 19, wherein the pulse waveform sensing unit comprises a pressure sensor and a cavity.

28. The dual-modality system according to claim 27, wherein the pressure sensor is made by organic flexible piezoelectric materials including polyvinylidene difluoride (PVDF) and has a serpentine structure to withstand bending and tensile deformation.

29. The dual-modality system according to claim 27, wherein the cavity is made by a high stiffness silicon rubber including PDMS, the cavity is located on the rectangular pressure sensor beam to form a piezo cantilever to improve force sensitivity of the pressure sensor.

30. The dual-modality system according to claim 19, wherein the entire system is encapsulated by a silicone rubber with modulus on par with that of human skin to provide a barrier to moisture for protecting the system from sweat corrosion, to allow conformation to different surfaces, and to endure twisting and stretching.
